(19) United States
(12) Patent Application Publication
(10) Pub. No.: US 2007/0263768 A1
(43) Pub. Date: Nov. 15, 2007

(54) Multimodality X-ray imaging

(76) Inventors: Christer Ullberg, Sollentuna (SE); Tom Francke, Sollentuna (SE)

Correspondence Address: HARNESS, DICKEY & PIERCE, P.L.C., P.O. BOX 8910, RESTON, VA, 20195, US

(21) Appl. No.: 11/472,275

(22) Filed: Jun. 22, 2006

(30) Foreign Application Priority Data
May 12, 2006 (SE) 0601068-0

(51) Int. Cl. G01N 23/04; A61B 8/00

(52) U.S. Cl. 378/63; 600/437

(57) ABSTRACT

The invention relates to a detector arrangement 1 for imaging an object. The detector arrangement 1 comprises a support device 4, an X-ray source 3 and an X-ray detection device 2 attached to the support device 4 on opposite sides of the object 8. The X-ray detection device 2 is arranged to detect radiation transmitted by the X-ray source 3 for producing an X-ray image. The detector arrangement 1 further comprises an ultrasonography device 9 for sending ultrasound pulses into the object 8 and for receiving reflections of the pulses for producing an ultrasonographic image. The ultrasonography device 9 is attached to the support device 4. The X-ray detection device 2 and the ultrasonography device 9 are arranged to image the object 8 in a single scanning movement.

### PRIORITY CLAIM

A claim of priority is made under 35 USC § 119 to Swedish Patent Application No. 0601068-0, filed on May 12, 2006, the contents of which are herein incorporated by reference in their entirety.

### FIELD OF THE INVENTION

The invention relates to the field of scanning-based detectors, and in particular to a detector arrangement for imaging an object.

### BACKGROUND OF THE INVENTION

It is often necessary to examine an object, for example in order to diagnose medical illnesses of patients or in order to check luggage at an airport. There are also various methods available for performing such examinations of objects, for example by imaging the object. Each method has its strengths and weaknesses and is consequently suitable for different situations. An examination may require anything from a few minutes up to several hours depending on type of examination. However, an examination requiring a lot of time is undesirable in view of efficiency, irrespective of application. Particularly when used in a medical application a prolonged examination is very trying for a patient undergoing the examination. Further, should an examination fail in any way, for example by providing images of insufficient quality, the procedure has to be repeated.

Further yet, although a specific method may be most suitable and therefore preferred for a particular application, there are situations when the chosen method can prove to be inadequate. For example, although mammography diagnostic procedure is one of the best methods for detecting early forms of breast cancer, it may still happen that a radiologist or physician reviewing the mammograms misses the detection of a tumour. Breast cancer can, for example, be missed by being obscured by radiographically dense, fibrograndular breast tissue.

An additional examination can in some cases be performed, for example if there are uncertainties in the obtained results. Then another examination method is preferably utilized, such as for example tomosynthesis imaging. However, to perform several examinations is very time consuming for the diagnostician as well as for the patient. Further, it is readily understood that this is most uncomfortable and unpleasant for a patient.

In view of the above it would be desirable to provide an improved way of examining objects, wherein the time required for an examination is minimized. Further, it would be desirable to increase the reliability of the results of an examination method while still minimizing the duration of the examination. In medical applications, it would be desirable to be able to also minimize the discomfort for a patient undergoing the examination.

### SUMMARY OF THE INVENTION

It is an object of the present invention to provide an arrangement for obtaining results from at least two different examination methods in a single examination session, thereby overcoming or at least alleviating the above-mentioned drawbacks of the prior art.

It is another object of the invention to provide an increased efficiency for obtaining the results from various examination methods, for example in terms of the time required to perform the respective examination methods.

It is still another object of the present invention to provide, when utilized in a medical application, examination means giving a lessened discomfort for a patient.

It is yet another object of the invention to enable two or more examinations to be performed without moving the object between the examinations, to thereby enable easy comparisons of the different images or even superimpose them on top of one another.

These objects, among others, are achieved by an arrangement as claimed in claim 1.

In accordance with the invention a detector arrangement for imaging an object is provided. The detector arrangement comprises a support device, an X-ray source and an X-ray detection device attached to the support device on opposite sides of the object to be examined. The X-ray detection device is arranged to detect radiation transmitted by the X-ray source for producing an X-ray image. The detector arrangement further comprises an ultrasonography device for sending ultrasound pulses into the object and for receiving reflections of the pulses for producing an ultrasonographic image. The ultrasonography device is attached to the support device. The X-ray detection device and the ultrasonography device are arranged to image the object in a single scanning movement. In accordance with the invention a single examination provides the results from different kinds of imaging methods. Should one of the methods turn out to give insufficient results then the results from the other diagnostic method can be utilized, or the results can complement each other. The inventive arrangement can be utilized in medical applications, in security application and in industrial applications. When utilized in a medical application, the invention provides lessened discomfort for a patient, since the examinations are performed almost simultaneously, without he patient having to be moved. This has also the advantage that the images can easily be compared to one another, or even superimposed on top of one another where each examination provides different information to be compared to one another.

In an embodiment of the invention, the X-ray detection device is arranged, in a scanning direction, ahead of and alongside the ultrasonography device. Alternatively, the ultrasonography device is arranged, in a scanning direction, ahead of and alongside the X-ray detection device. Flexibility is thereby provided, enabling different layouts of the arrangement, which thus can be adapted as best suited for a particular application.

In another embodiment of the invention, the arrangement comprises a third imaging device. The third imaging device is in an embodiment a device for performing a single photon emission computed tomography, and in another embodiment a device for performing position emission tomography. This provides an even more reliable examination to be performed in a single session.

In yet another embodiment of the invention, the object is a breast and the arrangement is arranged for use in mammography. This is an examination being widely performed, and the invention provides an increased reliability, without increased discomfort for the patient.

In an embodiment of the invention, the support device is an E-arm. This is a commonly used support device for X-ray examinations and is most suitable for the present invention, and readily available.

In yet another embodiment of the invention, the arrangement comprises a second ultrasonography device arranged perpendicular to the ultrasonography device. Three-dimensional images can thereby be obtained.

In still another embodiment of the invention, the arrangement further comprises a collimator arranged in front of the X-ray source. The collimator is preferably movable to simplify positioning of the patient. The object being examined is thereby subjected to a reduced radiation dose, which is particularly advantageous when examining a patient.

Further characteristics, advantages and objects of the invention will become apparent by reading the following detailed description.

### BRIEF DESCRIPTION OF THE DRAWINGS

FIG. 1 illustrates an arrangement in accordance with the present invention.

### DETAILED DESCRIPTION OF PREFERRED EMBODIMENTS

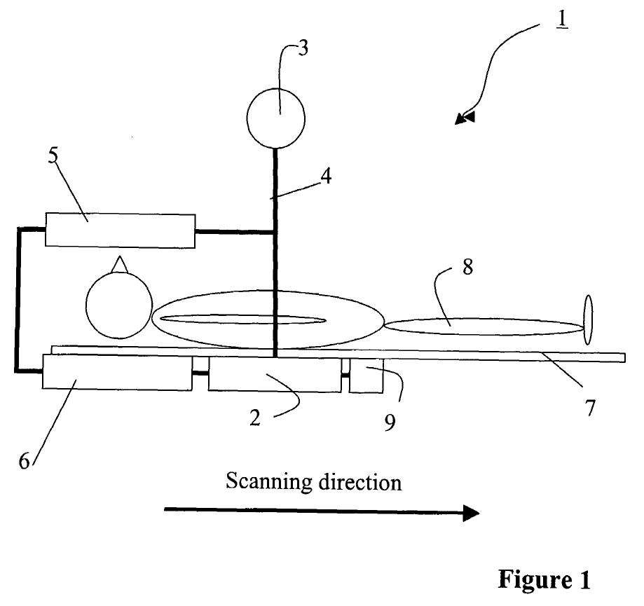

The present invention will now be described with reference to FIG. 1, which illustrates schematically an arrangement in accordance with the invention. The arrangement comprises a radiation or X-ray source arrangement 3 and an X-ray radiation detector device 2. A collimator arrangement (not shown) is preferably also included in order to reduce the radiation dose subjected to a patient.

The radiation source arrangement 3 preferably comprises one or more X-ray tubes having a cathode, which emits electrons, and an anode, which emits X-rays in response to being struck by the electrons. The radiation source arrangement 3 may further comprise a filter arrangement (not shown) just beneath the X-ray tubes, which typically includes thin metallic foils acting as filters to absorb the lowest (and sometimes also the highest) energy photons, which do not contribute significantly to the image quality. Preferably, if several X-ray tubes are included, such filter arrangement has different filter sections in front of the different X-ray tubes so that different radiation from different X-ray tubes may be filtered differently. This filter arrangement is typically optional, but may be required to provide radiation of different energies if two radiation sources are included emitting radiation of similar energy. The filter arrangement may alternatively be placed in or on a collimator. Reference is made to our pending U.S. patent application Ser. No. 10/442,208, the contents of which being hereby incorporated by reference.

The collimator, which is optional, may be a thin foil of e.g. tungsten with narrow radiation transparent slits etched away. The slits are aligned with corresponding line-shaped sensitive areas or entrance slits of the X-ray detector device 2 so that X-ray bundles passing through the slits of the fan beam collimator will reach the sensitive areas of the X-ray detector device 2. Yet optionally, a further collimator may be arranged in front of the X-ray detector device 2 (i.e. downstream of an object to be imaged).

In an embodiment of the invention, the X-ray detector device 2 comprises a plurality of direction sensitive line detectors arranged in an array, each extending in a vertical direction in order to record one-dimensional images in the vertical direction. Each of the line detectors is preferably a gaseous-based ionization detector, wherein electrons freed as a result of ionization by ionizing radiation entered into the line detector are accelerated, and optionally avalanche amplified, in a direction essentially perpendicular to the direction of the entered ionizing radiation. Such line detector is referred to as a gaseous-based edge-on detector.

Such line detectors and arrays thereof are further described in the following U.S. Patents issued to Tom Francke at al.: U.S. Pat. Nos. 6,337,482; 6,477,223; 6,476,397; 7,016,458; 7,006,597; 6,940,942; 6,970,533; 6,856,669; 6,873,682; 6,784,436; 6,794,656; 6,818,901; 6,627,897; 6,627,897; and 6,522,722, as well as in references therein, all of which being hereby incorporated by reference.

However, alternatively each of the line detectors may be any of a scintillator-based detector, a PIN-diode array, a TFT array, a CCD array, a gaseous-based detector, a liquid-based detector, a solid-state detector, or a CMOS detector.

Furthermore, the arrangement 1 comprises a microprocessor or computer (not shown) provided with suitable software for controlling the arrangement 1 and readout and post-processing of the data recorded by the X-ray detector device 2. Further, a power supply (not shown) is included for supplying the X-ray detector device 2 and the microprocessor or computer with power.

The X-ray source arrangement 3, optionally comprising a collimator, and the X-ray detector device 2 are, in the embodiment shown in the figure, attached to a support device, for example a common E-arm 4. It is realized that any other suitable support device can be utilized in alternative embodiments.

An object table 7 or the like can be part of the arrangement 1, and is then preferably arranged so as to enable a relative movement between the object being examined and the detector device 2. Alternatively, the arrangement 1 may be adapted to receive an object table.

In accordance with the invention, the arrangement 1 further comprises an ultrasonography device 9. Medical ultrasonography, or simply sonography, is an ultrasound-based diagnostic imaging technique and is often used for imaging internal organs of a patient. Ultrasonography is prevailingly utilized for scanning organs and provides a reliable method for diagnosing purposes.

The ultrasonography device 9 comprises a probe, also called scan head, containing one or more acoustic transducers for sending pulses of sound into the object that is being examined. The transducers are capable of generating and detecting ultrasound waves, and are constructed so that ultrasound beams are generated, followed by a pause during which the return waves are detected. This cycle continues during the entire diagnostic procedure. In an embodiment of the invention an array of acoustic transducers are utilized for constructing an image. An ultrasound beam is scanned or swept over the object and a two dimensional image is thereby obtained in a conventional manner. That is, transmitting a sound wave, determining which transducer elements receives the echo or reflection, the strength of the reflection and the time elapsed for the reflection to be received from when the sound was transmitted. The received signals are transformed into a digital image in a way that is known per se to a person skilled in the art. The ultrasonography device 9 thus comprises means for performing these calculations, such as signal processing electronics, which can be of a conventional type.

In an alternative embodiment, three dimensional ultrasonography images are provided. This can for example be accomplished by attaching a second ultrasonography device (not shown) oriented perpendicular to the first ultrasonography device 9.

A shortcoming of ultrasound devices is their difficulties to penetrate bones. The usefulness of ultrasound imaging is therefore limited in some situations, for example for imaging the brain of a patient. In view of this, in an alternative embodiment of the invention the different diagnostic methods are arranged to scan different parts of the body of a patient.

In yet another embodiment of the invention, the arrangement 1 further comprises means 5, 6 for performing a nuclear medicine tomographic imaging. For example, the arrangement may comprise a device for performing single photon emission computed tomography (SPECT). In an alternative embodiment, a device for performing positron emission tomography (PET) is included. The nuclear medicine tomographic imaging devices 5, 6 are also attached to the E-arm 4.

When the E-arm 4 of the arrangement 1 is moved relative the object 8 images are obtained in the same scanning movement as the X-ray images and ultrasonographic images. Such nuclear medicine tomographic imaging techniques enable tumours and bones, among other things, to be imaged. When performing a nuclear medicine imaging it is important that the patient does not move during the scan time, since movements can cause significant degradation of the reconstructed images. The arrangement 1 is thus brought to a halt when the nuclear medicine imaging is to be performed. When the imaging is completed, the E-arm 4 continues its scan movement.

In the illustrated embodiment, the detectors are shown as attached in a certain order; as seen in the indicated scanning direction the ultrasonography device 9 is placed first, thereafter the X-ray detection device 2 and lastly devices 5, 6 for nuclear medicine tomographic imaging. Any other attachment order is conceivable, for example the ultrasonography device 9 could be placed, as seen in the scanning direction, after the X-ray detection device 2 so that X-ray images are taken before the ultrasonographic images.

Further, the ultrasonography device 9 is shown in the figure as placed on the same side of the object as the X-ray detector device 2; however, in an alternative embodiment the ultrasonography device is placed on the same side of the object 8 as the X-ray source 3. It is realized that the placement of the ultrasonography device 9 is made such that it enables the two examination methods to be executed in a single scanning movement.

Further yet, in an alternative embodiment, the ultrasonography device 9 is arranged to measure the transmission of ultrasound pulses through the object. An ultrasound detector device (not shown) is then placed on the opposite side of the object for detecting ultrasound pulses transmitted by the ultrasonography device 9 and passing through the object.

The arrangement 1 may comprise a movement device for moving the X-ray detection device 2 and the ultrasonography device 9 relative the object 8 that is being examined. However, it shall be appreciated that the arrangement may be modified such that the object 8 is moved during scanning, while the E-arm 4 comprising the detectors 2, 5, 6 and 9 are kept at rest.

In the figures the object 8 being examined is illustrated as being a patient, but it is realized that other applications are also conceivable. The inventive arrangement may for example be utilized in security applications such as scanning luggage or in industrial application such as performing material testing or the like. It may, in many situations, be beneficial to obtain images of an object by utilizing several different examination methods and the invention provides a simple means for achieving this. In the preceding description a medical application has been described. However, it is realized that the invention may be utilized in other applications as well. For example, by imaging an object in different ways an increased security can be obtained at an airport.

In accordance with the invention, several examination methods can thus be performed at a single examination occasion. Therefore, should it be necessary to perform an additional examination, utilizing another examination method, the patient does not have to be moved. As an X-ray examination can be performed simultaneously with a medical ultrasonic diagnostic imaging, most reliable results are obtained in a very efficient way. Should one of the methods turn out to give insufficient results then the results from the other diagnostic method can be utilized. Another advantage is that the patient does not have to be moved from one examination room to another. Today, this is typically the case if different examination methods are to be used; the patient often even has to be moved between different wards of a hospital. This is very time consuming for the diagnostician as well as for the patient and is avoided by means of the invention. The discomfort experienced by a patient is thereby minimized.

In summary, the present invention provides an arrangement for obtaining imaging results from at least two different examination methods. The results are preferably obtained in an examination performed in a single scanning, whereby the patient does not have to be moved between different types of examinations or different examination rooms. Further, by performing several kinds of examinations in one examination procedure, the examination undergone causes the least possible discomfort for a patient. By means of the invention, an object can be imaged by the use of several different examination methods within a short period of time and by utilizing a single arrangement.

It shall be appreciated that the various embodiments of the present invention may be combined to reach still further embodiments of the invention. Various features and details as specified in some of the embodiments of the invention may be equally applicable in other ones of the embodiments.

1. A detector arrangement for imaging an object wherein the arrangement comprises:
a support device,
an X-ray source and an X-ray detection device attached to said support device on opposite sides of said object, said X-ray detection device being arranged to detect radiation transmitted by said X-ray source for producing an X-ray image and
an ultrasonography device for sending ultrasound pulses into said object and for receiving reflections of said pulses for producing an ultrasonographic image, said ultrasonography device being attached to said support device, wherein
said X-ray detection device and said ultrasonography device are arranged to image the object in a single scanning movement.

2. The arrangement as claimed in claim 1, wherein said X-ray detection device is arranged, in a scanning direction, ahead of and alongside said ultrasonography device.

3. The arrangement as claimed in claim 1, wherein said ultrasonography device is arranged, in a scanning direction, ahead of and alongside said X-ray detection device.

4. The arrangement as claimed in claim 1, wherein said arrangement comprises a third imaging device.

5. The arrangement as claimed in claim 4, wherein said third imaging device is a device for performing a single photon emission computed tomography.

6. The arrangement as claimed in claim 4, wherein said third imaging device is a device for performing positron emission tomography.

7. The arrangement as claimed in claim 1, wherein said object is a breast and said arrangement is arranged for use in mammography.

8. The arrangement as claimed in claim 1, wherein said support device is an E-arm.

9. The arrangement as claimed in claim 1, wherein said scanning movement is a linear movement.

10. The arrangement as claimed in claim 1, wherein said arrangement comprises a second ultrasonography device arranged perpendicular to said ultrasonography device, whereby three-dimensional images are obtained.

11. The arrangement as claimed in claim 1, wherein said arrangement further comprises a collimator arranged in front of said X-ray source.

12. The arrangement as claimed in claim 11, wherein said collimator is a thin foil with narrow radiation transparent slits etched away.

13. The arrangement as claimed in claim 1, wherein said arrangement further comprises a movement device for moving said X-ray detection device and said ultrasonography device relative said object.

14. The arrangement as claimed in claim 1, wherein said X-ray detection device comprises means for providing one-dimensional images.

15. The arrangement as claimed in claim 1, wherein said ultrasonography device comprises means for providing one-dimensional images.

16. The arrangement as claimed in claim 14, wherein said X-ray detection device and said ultrasonography device are arranged to provide two-dimensional images during said scanning movement.

17. The arrangement as claimed in claim 1, wherein the X-ray detector device comprises a plurality of direction sensitive line detectors arranged in an array.

18. The arrangement as claimed in claim 17, wherein each of said line detectors is a gaseous-based ionization detector.
